(19) United States
(12) Patent Application Publication
(10) Pub. No.: US 2014/0121256 A1
(43) Pub. Date: May 1, 2014

(54) METHOD OF TREATING MUSCULAR WEAKNESS COMPRISING ADMINISTERING A COMPOSITION COMPRISING AN EFFECTIVE AMOUNT OF HISTAMINE AND/OR SEROTONIN

(71) Applicant: BEECH TREE LABS, INC., Delanson, NY (US)

(72) Inventor: John McMichael, Delanson, NY (US)

(73) Assignee: BEECH TREE LABS, INC., Delanson, NY (US)

(21) Appl. No.: 14/062,683

(22) Filed: Oct. 24, 2013

Related U.S. Application Data
(60) Provisional application No. 61/718,933, filed on Oct. 26, 2012.

(51) Int. Cl. A61K 31/417; A61K 31/4045

(52) U.S. Cl. CPC A61K 31/417; A61K 31/4045
USPC 514/400

(57) ABSTRACT

The present invention relates to a treatment of muscle weakness resulting from a disease state or injury in a subject suffering therefrom by administering a composition including histamine in an amount effective to alleviate muscle weakness in the subject suffering therefrom.

### CROSS-REFERENCE TO RELATED APPLICATIONS

This application claims priority benefit of U.S. Provisional Application 61/718,933 filed Oct. 26, 2012 the disclosure of which is incorporated herein by reference.

### BACKGROUND OF THE INVENTION

The present invention is related to methods for treating muscle weakness resulting from both muscle weakness inducing disease states and injuries to the muscle tissue which result in muscle weakness. Muscle weakness can result as the consequence of a physical trauma to the muscle or surrounding tissue as well as from disease. Diseases resulting in muscle weakness include but are not limited to those such as muscular dystrophy, post-polio syndrome, multiple organ dysfunction syndrome, myasthenia gravis, chronic fatigue syndrome, and inflammatory myopathy.

Muscular dystrophy (MD) is a group of muscle diseases that weaken the musculoskeletal system and hamper locomotion. Muscular dystrophies are characterized by progressive skeletal muscle weakness, defects in muscle proteins, and the death of muscle cells and tissue. In the 1860s, descriptions of boys who grew progressively weaker, lost the ability to walk, and died at an early age became more prominent in medical journals. The most common form of MD is labeled as Duchene muscular dystrophy (DMD) for the French neurologist Guillaume Duchenne. The other major forms are Becker, limb-girdle, congenital, facioscapulohumeral, myotonic, oculopharyngeal, distal, and Emery-Dreifuss muscular dystrophy. These diseases predominately affect males, although females may be carriers of the disease gene. Most types of MD are multi-system disorders with manifestations in body systems including the heart, gastrointestinal system, nervous system, endocrine glands, eyes and brain.

Apart from the nine major types of muscular dystrophy listed above, several MD-like conditions have also been identified. Normal intellectual, behavioral, bowel and sexual function are noticed in individuals with other forms of MD and MD-like conditions. MD-affected individuals with susceptible intellectual impairment are diagnosed through molecular characteristics but not through problems associated with disability. However, a third of patients who are severely affected with DMD may have cognitive impairment, behavioral, vision and speech problems.

These conditions are generally inherited, and the different muscular dystrophies follow various inheritance patterns. However, mutations of the dystrophin gene and nutritional defects (with no genetics history) at the prenatal stage are also possible in about 33% of people affected by DMD. The main cause of the Duchenne and Becker types of muscular dystrophy is the muscle tissue's cytoskeletal impairment to properly create the functional protein dystrophin and dystrophin-associated protein complex.

Dystrophin protein is found in muscle fiber membrane, acting like a spring. It joins the membrane actin filaments. The hydrophobic protein is rod shaped around 150 nm in length, 3684 amino acids long, 427 kDa molecule weight and has an alpha-helical conformation allowing protein to act as a shock absorber, preventing overstress. Dystrophin links actin (cytoskeleton) and dystroglycans of the muscle cell plasma membrane, known as the sarcolemma (extracellular). Dystrophin functions in two ways; mechanical stabilization and regulated calcium levels. The diagnosis of muscular dystrophy is based on the results of muscle biopsy, increased creatine phosphokinase (CpK3), electromyography, electrocardiography and DNA analysis.

The prognosis for people with muscular dystrophy varies according to the type and progression of the disorder. Some cases may be mild and progress very slowly over a normal lifespan, while others produce severe muscle weakness, functional disability, and loss of the ability to walk. Some children with muscular dystrophy die in infancy while others live into adulthood with only moderate disability. The muscles affected vary, but can be around the pelvis, shoulder, face or elsewhere. Muscular dystrophy can affect adults, but the more severe forms tend to occur in early childhood.

There is no known cure for muscular dystrophy, although significant headway is being made with antisense oligonucleotides. Physical therapy, occupational therapy, orthotic intervention (e.g., ankle-foot orthosis), speech therapy and orthopedic instruments (e.g., wheelchairs and standing frames) may be helpful. Inactivity (such as bed rest, sitting for long periods) and bodybuilding efforts to increase myofibrillar hypertrophy can worsen the disease.

Occupational therapy assists the individual with MD in engaging in his/her activities of daily living (self-feeding, self-care activities, etc.) and leisure activities at the most independent level possible. This may be achieved with use of adaptive equipment or the use of energy conservation techniques. Occupational therapy may implement changes to a person's environment, both at home or work, to increase the individual's function and accessibility. Occupational therapists also address psychosocial changes and cognitive decline which may accompany MD, and provide support and education about the disease to the family and individual.

There is no specific treatment for any of the forms of muscular dystrophy. Physiotherapy, aerobic exercise, low intensity anabolic steroids, prednisone supplements may help to prevent contractures and maintain muscle tone. Orthoses (orthopedic appliances used for support) and corrective orthopedic surgery may be needed to improve the quality of life in some cases. The cardiac problems that occur with Emery-Dreifuss muscular dystrophy and myotonic muscular dystrophy may require a pacemaker. The myotonia (delayed relaxation of a muscle after a strong contraction) occurring in myotonic muscular dystrophy may be treated with medications such as quinine, phenytoin, or mexiletine, but no actual long term treatment has been found.

Post-polio syndrome (PPS) affects from 25 to 50% of patients who have previously contracted poliomyelitis and presents with symptoms including acute or increased muscle weakness, muscle pain and fatigue. The disease is slowly progressive and its mechanism is unknown but the “neural fatigue” theory posits that motor neurons surviving attack by poliovirus form new nerve terminals to orphaned muscle fibers but that the enlargement of the motor neuron fibers places added metabolic stress on the nerve cell body which eventually leads to the deterioration of the sprouted muscle fibers and the neuron itself. Treatment consists mainly of palliative therapy and medications for fatigue such as amantadine and pyridostigmine and therapies such as prednisone and coenzyme Q10 have not been reported to be effective. Treatment with intravenous immunoglobulin (WIG) have shown modest positive results but there remains a need for effective treatments.

Multiple Organ Dysfunction Syndrome (MODS) can result from sepsis and septic shock which are life threatening conditions but can also result in muscle weakness.

Myasthenia gravis is a neuromuscular disease which can lead to muscle weakness and fatigue. The disease has an autoimmune component in which antibodies inhibit the effects of acetylcholine on nicotinic receptors throughout neuromuscular junctions. Medications such as immunosuppressive drugs and acetylcholinesterase inhibitors have proven useful in treatment but there remains a need for improved treatments.

Chronic Fatigue Syndrome (CFS) is a disorder characterized by persistent fatigue accompanied by other specific symptoms such as malaise, muscle and joint pain, mental and physical exhaustion and muscle weakness. It is reported that many people do not fully recover from CFS but treatments including cognitive behavioral therapy, graded exercise therapy and pacing can provide relief. Nevertheless, there remains a need for improved treatments for CFS.

Myopathy is a muscular disease in which muscle fibers do not function for various reasons resulting in weakness of the muscle. Inflammatory myopathy is a form of myopathy that involves inflammation of the muscle and includes three related diseases: polymyositis, dermatomyositis and inclusion-body myositis.

Crush injuries and other trauma can temporarily or more permanently injure muscle tissue resulting in weakness. Crush injury syndrome, also known as rhabdomyolysis, is a condition in which skeletal muscle tissue breaks down rapidly. Breakdown products of the injured muscle are released into the bloodstream where they can cause kidney damage or even failure but also lead to muscle pains and weakness vomiting and confusion. Treatment focuses on preserving kidney function and addressing other complications such as compartment syndrome such as by surgery to relieve pressure inside the muscle compartment and reduce the risk of compression on blood vessels and nerves in the area. There remains a need for improved therapies for rehabilitating skeletal muscle.

### BRIEF SUMMARY OF THE INVENTION

The invention provides methods for treating muscle weakness resulting from a disease state or injury in a subject suffering therefrom comprising the step of: administering a composition comprising histamine in an amount effective to alleviate muscle weakness in the subject suffering therefrom. Disease states or injuries susceptible to treatment include those wherein the disease state or injury is one selected from the group consisting of muscular dystrophy, post-polio syndrome, multiple organ dysfunction syndrome, myasthenia gravis, chronic fatigue syndrome, crush injury and inflammatory myopathy.

Various amounts of histamine can be administered but the composition preferably comprises from about 4×10⁻¹ to about 4×10⁻⁵ mg of histamine; more preferably from about 4×10⁻² to about 4×10⁻⁴ mg of histamine and most preferably about 1×10⁻³ mg of histamine. Different forms of histamine can be used but histamine is preferably used in the form of a soluble salt such as histamine phosphate.

The compositions of the invention can further comprise from about 0.2 mg to about 2×10⁻⁶ mg of serotonin (5-hydroxytryptamine); more preferably from about 2×10⁻² mg to about 2×10⁻⁴ mg of serotonin and most preferably about 2×10⁻³ mg of serotonin. Thus, the compositions of the invention can comprise the administration of both histamine and serotonin in combination with a preferred composition comprising 4×10⁻² to about 4×10⁴ mg of histamine and from about 2×10⁻² mg to about 2×10⁻⁴ mg of serotonin. Most preferred is a composition comprising about 0.08 mg of histamine and about 4×10⁻³ mg of serotonin.

According to one preferred aspect of the invention the composition is administered in a single dose of about 0.05 cc in a pharmaceutically acceptable carrier four times daily. The composition may be administered by various modes but a particularly preferred mode is sublingual administration.

### BRIEF DESCRIPTION OF THE DRAWINGS

FIGS. 1A and 1B depicts the results of various compositions on tetanic force assay with a “1×” composition defined as providing a dosage of 4.8×10⁻³ mg histamine and 0.08 milligram serotonin.

FIG. 2 depicts the results of a composition including histamine and serotonin in a tetanic force assay.

FIG. 3 depicts the results of a composition including histamine and serotonin at various concentrations in a contraction induced injury assay.

FIG. 4A depicts the results of a composition including histamine and serotonin (MDX) versus untreated wild-type versus a phosphate buffered saline (PBS) control in a mouse triangle force assay; FIG. 4B depicts the results of a composition including histamine and serotonin (MDX) versus untreated wild-type versus a phosphate buffered saline (PBS) control in a mouse mesh force assay; and FIG. 4C depicts the results of a composition including histamine and serotonin in a mouse wire hand test.

FIG. 5A depicts the results of a composition including histamine and serotonin at different dosages in a mouse triangle force assay; and FIG. 5B depicts the results of a composition including histamine and serotonin at different dosages in a mouse mesh force assay.

FIG. 6A depicts the results of a composition including histamine alone at different dosages in a mouse triangle force assay; and FIG. 6B depicts the results of a composition including histamine alone at different dosages in a mouse mesh force assay.

FIG. 7A depicts the results of a composition including serotonin alone at different dosages in a mouse triangle force assay; and FIG. 7B depicts the results of a composition including serotonin alone at different dosages in a mouse mesh force assay.

FIG. 8A depicts the results of a composition including histamine and serotonin at different dosages in a mouse triangle force assay; and FIG. 8B depicts the results of a composition including histamine and serotonin at different dosages in a mouse mesh force assay.

### DETAILED DESCRIPTION OF THE INVENTION

The invention provides the use of histamine to successfully treat muscle weakness. The histamine used according to the methods of the invention is preferably in the form of a soluble salt such as histamine phosphate. The preferred concentration of histamine base in each dose of the invention is generally within the range of about 4×10⁻¹ to about 4×10⁻⁵ mg. More preferably, the concentration of histamine base in each dose of the invention is generally within the range of about 4×10⁻² to about 4×10⁻⁴ mg and most preferably about 1×10⁻³ mg, of histamine per dose.

Serotonin can also be incorporated into the treatment compositions with preferred amount of the serotonin in each dose of the invention contains from about 0.2 mg to about 2×10⁻⁶ mg of serotonin (5-hydroxytryptamine). More preferably, the dosage of serotonin is generally within the range of about 2×10⁻² mg to about 2×10⁻⁴ mg and most preferably about 2×10⁻³ mg of serotonin.

Methods for administering the present invention to a patient suffering from muscle weakness vary and may include, inter alia, administration subcutaneously, interperitoneally, intravenously, intramuscularly, or sublingually, with sublingual administration being particularly preferred.

According to the present invention, histamine and serotonin may be combined in a single composition or may be administered individually. Generally, a patient begins treatment by sublingually administering one drop four times daily, with each drop being equivalent to about 0.05 cc. The number of drops may vary daily but a regimen in which four drops are administered daily is preferred.

Provided below are case histories of patients diagnosed with symptoms of muscle weakness. The histamine used in these trials was obtained from Allermed Laboratories. The serotonin used was 5-hydroxytryptamine obtained from Sigma.

### Example 1

According to this example, a composition comprising histamine and serotonin was administered at 6 different concentrations on normal human BioArtificial Muscles (mBAMs engineered from human skeletal rmyoblasts) for effects on strength (active force generation) and injury using Myomics' MyoForce Analysis System (MFAS™).

Cell Culture

A solution comprising 200 times the dosage of the histamine and serotonin present in a typical therapeutic drop (i.e., 1 mg histamine and 16 mg serotonin in PBS) stored at 4° C. was administered to a cell culture comprising human skeletal muscle cells previously isolated from a normal (disease-free) 48 year old female were used for this study. Cells were expanded in culture in a Growth Medium optimized for human skeletal muscle (SKGM/15) for 5 days before engineering into bioartificial muscles (mBAMs). On the day of engineering, mBAMs, cells were trypsinized and counted following standard laboratory protocols.

Tissue-Engineering

A sterile 96 well MyoForce plate was used for tissue engineering the mBAMs. Myomics' Robotic Tissue Engineering Platform was used to engineer 72 mBAMs in approximately 10 minutes by mixing cells with an extracellular matrix solution and casting in the plate wells. The plate containing the mBAMs was maintained for 2 days at 37° C. in SKGM/15, then incubated in an optimized differentiation medium for the remainder of the experiment.

Electrical Stimulation for Active Force Measurements

On day 9 after plating, the mBAM plates were transferred to a Myomics' Myoforce Assay Device (MAD™). mBAMs were electrically stimulated by parameters set for generating maximal muscle contraction, i.e. tetanic (active) force. Each 96 well MyoForce plate in MAD™ takes approximately 15 minutes to stimulate all the wells. A high-speed imaging camera captures the images of the mBAMs as they contract, and sends the data to the computer system for calculating microNewtons (μN) of force generated. After the initial electrical stimulation, one drop (0.05 ml) of the composition comprising various concentrations of histamine and serotonin was added to each well to at final concentrations of 0.01×, 0.1×, 1.0×, 2.5×, 5×, and 10× (with 0.05 ml at 1.0× providing a dosage 4.8×10⁻³ mg histamine and 0.08 milligram serotonin) as specified by BTL. Other treatment groups included in the assay were Myomics' positive control and a vehicle-only control. Each test and control group was assayed with 8 replicates.

Every 24 hours thereafter for a total of 4 days, the mBAMs were again electrically stimulated for force measurements, and the medium replaced with fresh compound-containing medium.

Injury Assay

To assess the effect of compound histamine serotonin composition on muscle injury, mBAMs were subjected to repetitive tetanic electrical stimulations in the MAD™ after 4 days of drug treatment. Each mBAM received a total of 16 consecutive electrical pulses. Images were recorded during each electrical stimulation.

After the mBAMs had received the 16 consecutive electrical pulses, the effects of the histamine and serotonin composition on the contraction-induced injury were determined. ToxiLight BioAssay (Lonza) was used to measure the release of adenylate kinase (AK) through leakage from damage to the plasma membrane of the muscle cells. The ToxiLight

Assay measures the conversion of ADP to ATP in the presence of AK. The enzyme luciferase then catalyzes the formation of light from luciferin in the presence of ATP. The emitted light, measured with a luminometer, is linearly related to the AK concentration, and therefore is indicative of the extent of damage to the cell membrane. Conditioned medium was collected from the mBAMs after injury for assay of released AK, and mBAMs were then lysed, centrifuged, and the supernatant was collected to determine intracellular AK. The AK released upon injury was calculated as a percent of the total AK (released plus intracellular).

Results:

Strength (Active Force Measurements)

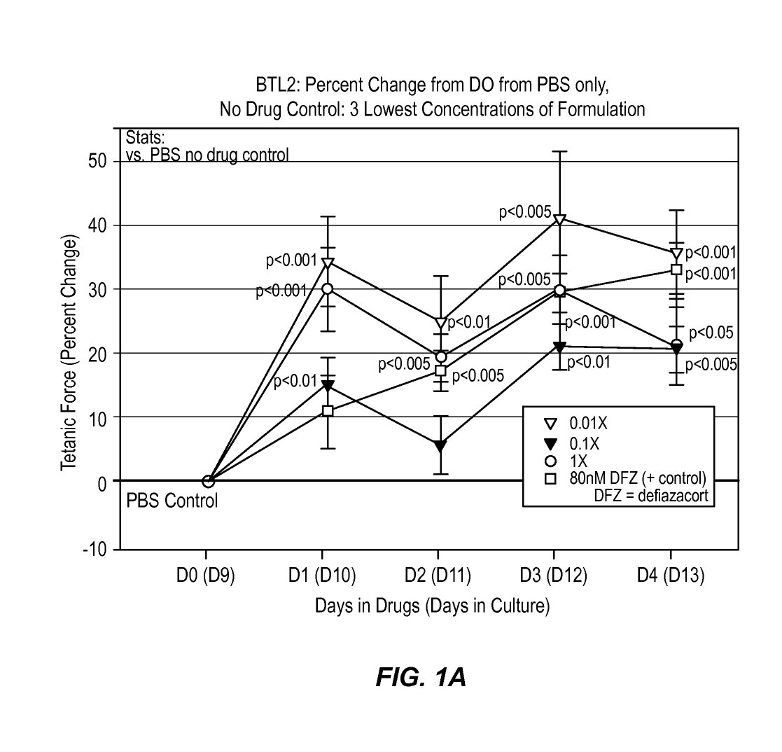

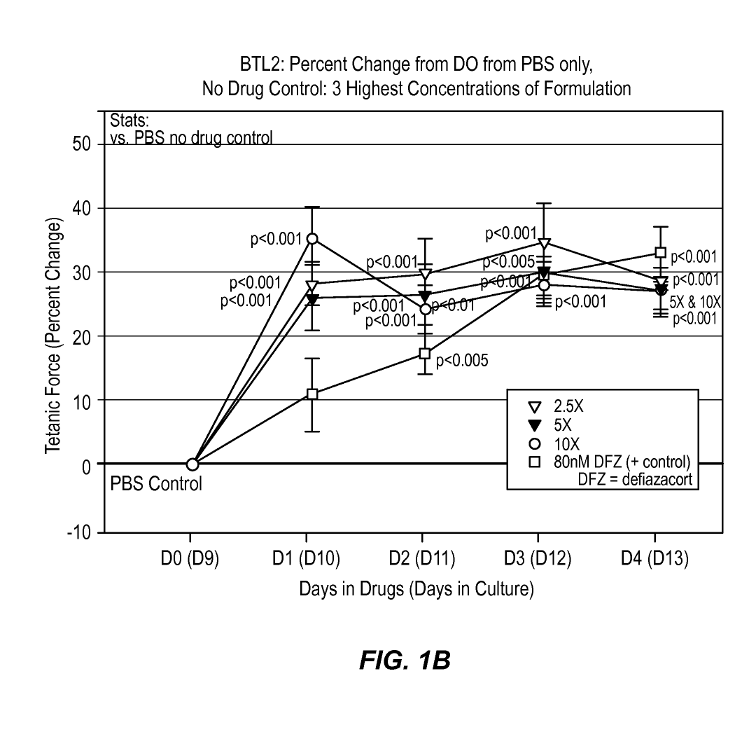

a. Time course of treatment with The inventive composition showed significant increases in tetanic forces at all time points, and at all doses tested compared to untreated controls, beginning on day one after compound addition [FIG. 1A (3 lowest doses) & 1B (3 highest doses)].

b. Myomics' positive control, 80 nM deflazacort (DFZ), showed a significant increase (32%; p<0.001) in tetanic force after 3-4 days of drug treatment compared to untreated controls, validating the strength assay. (FIG. 1)

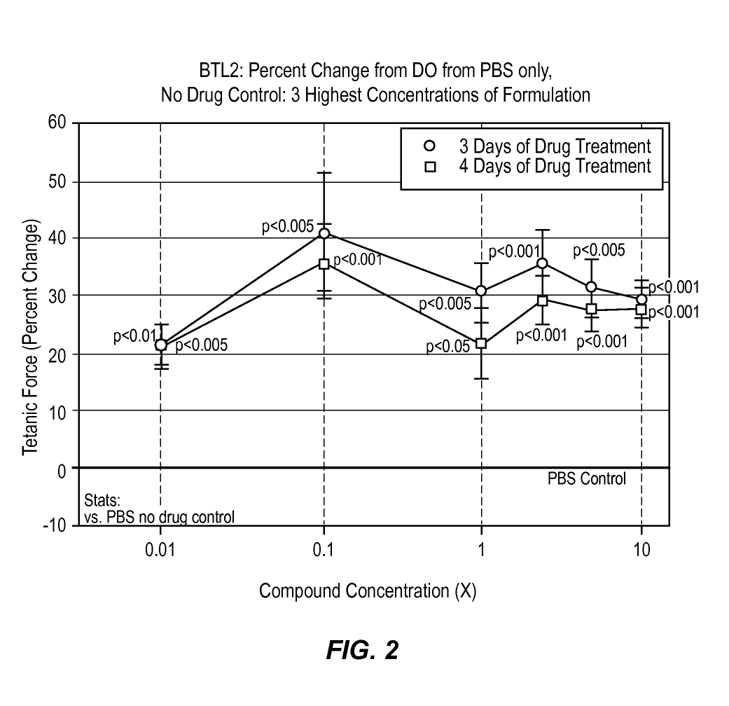

c. Dose response of mBAMs to The inventive composition showed a significant increase in force generation at all concentrations tested compared to no drug controls (PBS only) after 3-4 days of drug treatment, with increases ranging from 20.9 to 40.8%. Highest increases in force generation were seen with 0.1× (40.8% day 3; 35.7% day 4) and 2.5× (with a 0.05 ml dosage at 1.0× providing 4.8×10⁻³ mg histamine and 0.08 milligram serotonin) (35.0% day 3; 28.8% day 4) (FIG. 2).

Injury

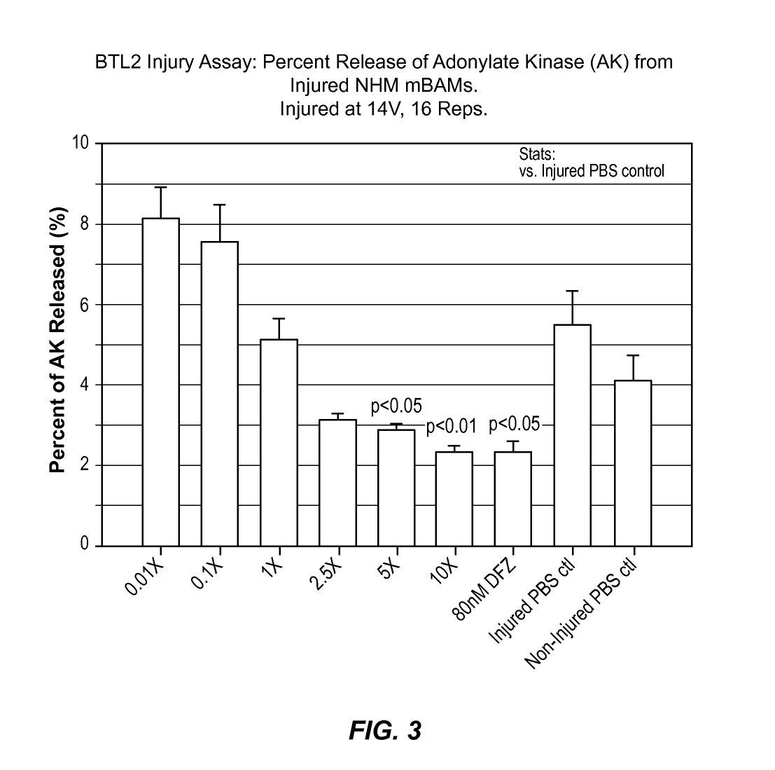

a. The inventive composition at concentrations of 5× & 10× (with a 0.05 ml dosage at “1.0×” being 4.8×10⁻³ mg histamine and 0.08 milligram serotonin and “5.×” “10×” being five and ten times concentration multiples thereof) had a significant effect on reducing injury to the mdx mBAMs as measured by a decrease in release of adenylate kinase by the ToxiLight assay compared to untreated controls (FIG. 3).

b. mBAMs treated with the histamine and serotonin compositions at 0.01×, 0.1×, 1.0× and 2.5× concentrations (with a 0.05 ml dosage at 1.0× providing 4.8×10⁻³ mg histamine and 0.08 milligram serotonin) and subjected to contraction induced injury as described, did not show a significant difference in AK release from untreated control BAMs (FIG. 3).

### Example 2

According to this example a subject suffering from Muscular Dystrophy of an unspecified type exhibiting muscle weakness was treated with a combination of 4.8×10⁻³ mg histamine and 0.08 mg serotonin administered sublingually, four times daily. The subject reported that her left side was still weaker than her right side of her body, that her arms are still weak and that she cannot hold yoga poses very long. In particular, her arms give out in downward positions, her trunk muscle was also weak as were her neck and upper back muscles.

After three weeks of treatment the subject reported improvements in muscle strength in which she was able to improve her performance on an elliptical trainer by 10-18% and was walking better and was happier.

She was able to roll over in her bed much better than before. Previously the covers felt too heavy on her body and had a suffocating effect that made it difficult to move. The subject could lift her head slightly off the pillow whereas before she could not do so unless she was propped up in bed.

While it was not clear that the subject was better able to climb stairs she was able to get up from the couch using momentum of her upper body without using her hands in flat shoes most times and was able to sometimes stand from sitting on a toilet without assistance.

She still required the use of her hands to stand from sitting when she wears high heels but this was easier for her. In addition, she was able to life her leg into a car without using her hands. These improvements have been maintained for fifteen months without plateauing.

### Example 3

According to this example a composition comprising histamine and serotonin was administered to a mouse model of Duchene's Muscular Dystrophy (DMD) in mice. Duchene's Muscular Dystrophy (DMD) is one of the more severe forms of Muscular Dystrophies (MD) that afflicts people. DMD is caused by one or more mutations in genes that produce the protein Dystrophin.

Female mouse strain C57BL/10SCSN-Dmd^{mdx}/J (Jackson laboratories) is a strain of mice that is most similar in affliction to humans with DMD. The mdx mutation of Dmd is recessive and heterozygous females are visually indistinguishable from wild-type mice. Like human patients who suffer from one of the most common neuromuscular diseases, Duchenne muscular dystrophy (DMD), the Dmd^{mdx} mutants do not express dystrophin and therefore have been routinely used as an animal model of the disease even though the resultant myopathology is much less severe compared to the human disease course. This strain of mice comes with a control strain, C57BL/10SC SnJ (Jackson laboratories), which is a wild type mouse that does not have the DMD mutations. One of the drawbacks of this mouse model is that by six weeks (10 weeks of age) the afflicted mice mimic the wild type control mice. This is attributed to the compensatory function of the dystrophin-related protein utrophin, which is highly up regulated in regenerating muscle fibers in adult Dmd^{mdx} mutants. The histamine and serotonin formulation was evaluated using this mouse model to determine its effects on DMD.

Preliminary Experiment:

The first experiment conducted focused on whether any difference could be detected in mice receiving the histamine and serotonin formulation vs. control. Only one dosage “1×”, was tested which provided 4.8×10⁻³ mg histamine and 0.08 milligram serotonin in a 0.1 ml dosage. The experiment was set up as follows: six control wild type mice, mice not afflicted with DMD were used as control for disease progression; ten mdx control mice, five per cage served as positive control; ten mdx experimental mice, five per caged served as experimental treatment animals.

Mice were four weeks of age when experimental basal measurements were taken. Injections started the following day. All mice were injected subcutaneously twice a day with 0.1 CC of solution 4.8×10⁻³ mg histamine and 0.08 milligram serotonin.

The injection site was located just below the neck on their dorsal side. Measurements to determine effectiveness of histamine and serotonin formulation were conducted using two different methods: 1. a wire hang test was utilized to see how long the mice were able to hang onto a wire before releasing and falling, and 2. a grip strength meter device (Columbus Instruments) was used to measure the peak force exerted by the animal when pulled.

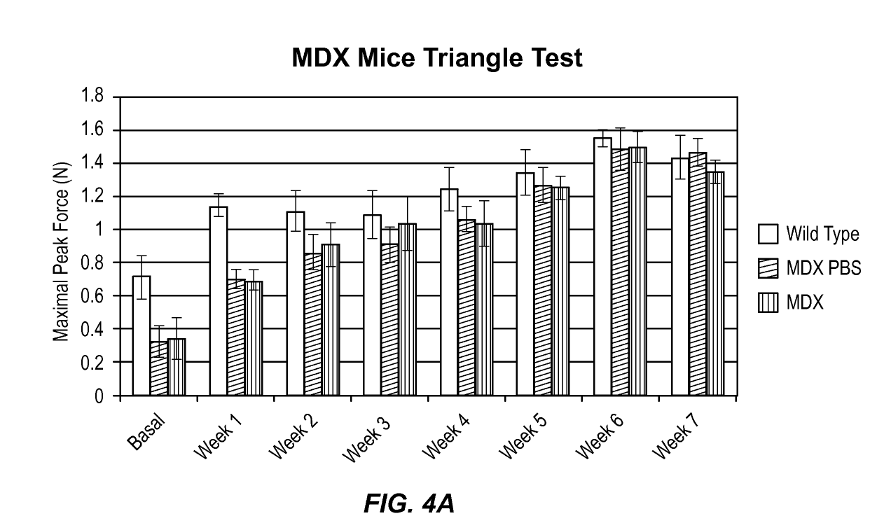

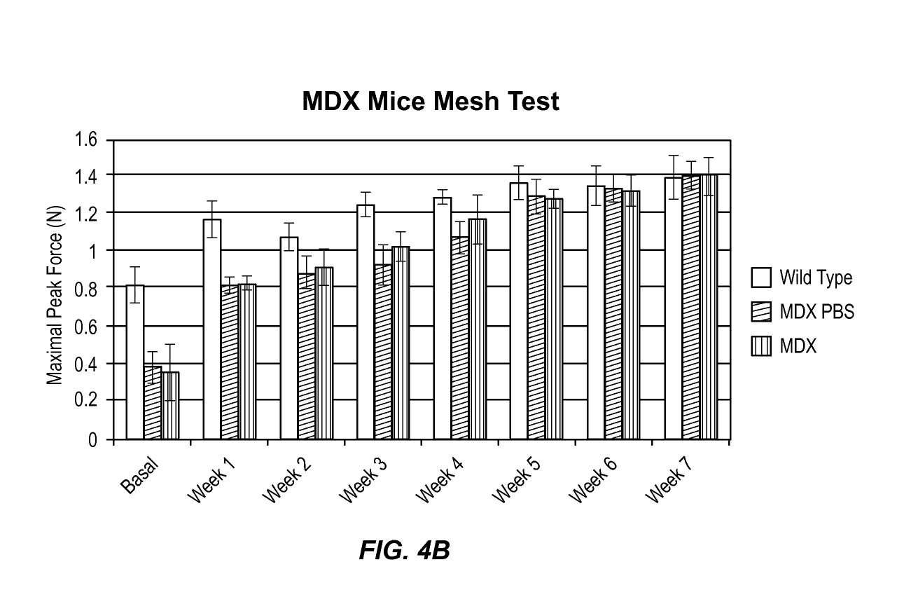

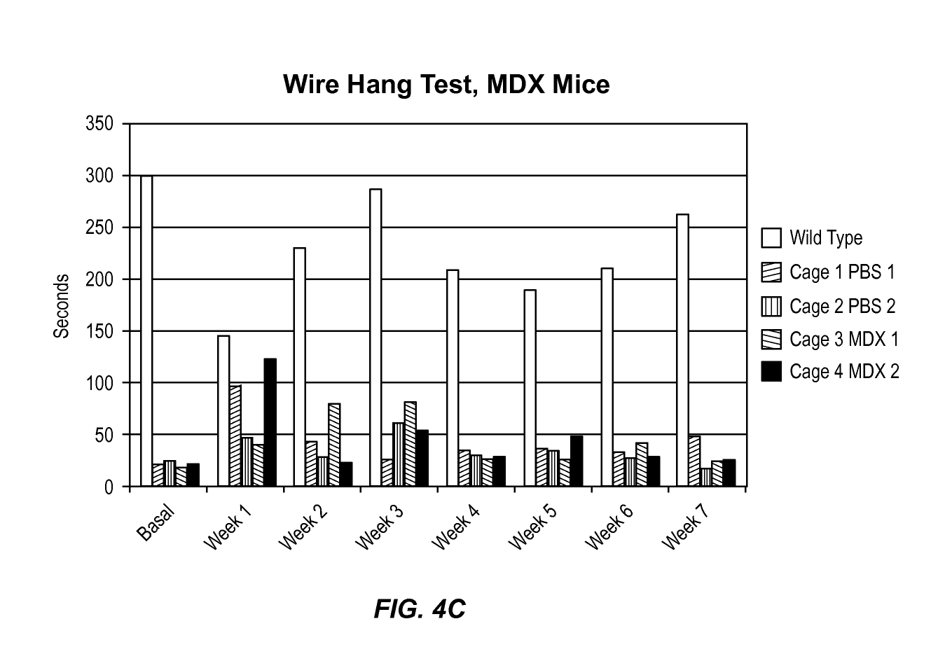

In the grip strength test, the mouse grips the bar with her front paws as the experimenter pulls her slowly away until she releases the bar. The number measured is the force required to remove the mouse. The higher the number the more force that is required to dislodge the mouse. This test is conducted using two types of grips; a triangle grip and a mesh grip. Each animal was pulled five times to determine the maximal force and the average was recorded. This was conducted for both the triangle and the mesh tests at once weekly interval for the duration of the experiment. For the wire hang test, the mice were allowed to hang for a maximum of five minutes each. This was only done once per animal at once weekly intervals for the duration of the experiment. The results of these tests are presented in FIGS. 4A-C

Preliminary results, while not statistically significant, show that the histamine and serotonin formulation appears to be trending towards improving muscle strength of the afflicted mice. This is seen in both the triangle test and mesh test at week two and three and in the mesh test at week four. The wire hang test was inconclusive, as some of the mice were able to pull themselves onto the wire and sit there in a perched position. While the data are trending toward the histamine and serotonin formulation as having some sort of effect, a more detailed second experiment was conducted to further evaluate this.

Follow-Up Experiment:

The second experiment conducted focused on whether any differences in histamine and serotonin formulation concentrations could enhance the results seen in the previous experiment. Histamine and serotonin formulations at the following concentrations were used: 0.4×, 1×, 4× and 8×. A 0.1 ml dosage at a 0.4× concentration contains 8.8 mcg histamine and 16 mcg serotonin per ml; 1× as previously described; 4× concentration contains 88 mcg histamine and 160 mcg serotonin per ml; 8× concentration contains 176 mcg histamine and 320 mcg serotonin per ml. The experimental setup was slightly different from the previous experiment and was as follows: Four control wild type mice, mice not afflicted with DMD were used to control for disease progression; Seven mdx control mice, which served as positive controls; and seven mdx experimental mice per cage, which served as experimental treatment animals (0.4×, 1×, 4× and 8×) with each cage treated with a different concentration of the histamine and serotonin formula.

All mice were injected subcutaneously twice a day with 0.1 CC of histamine and serotonin solution. The injection site was located just below the neck on their back. Mice were four weeks of age when experimental basal measurements were taken and injections started the same day. For both of the grip strength tests, ten measurements were taken per mouse and the average was recorded.

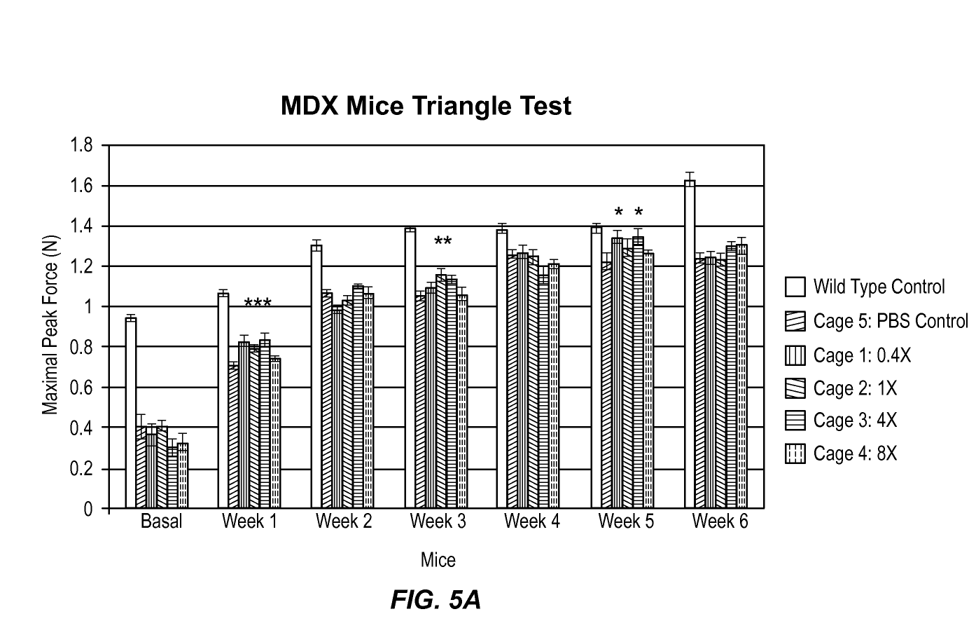

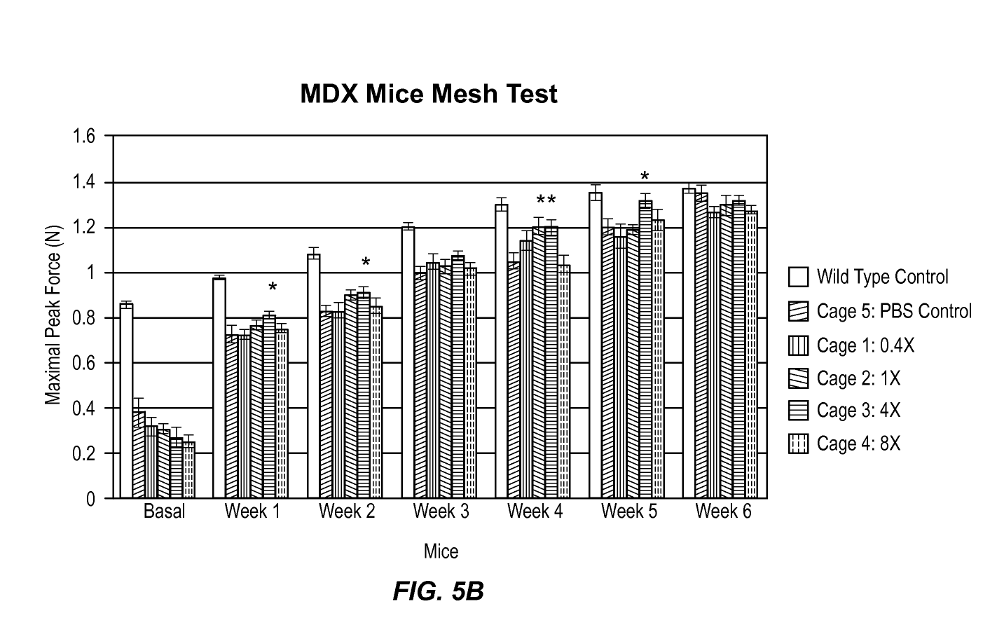

The results of the second experiment are depicted in FIGS. 5A and 5B. Samples with an asterisk above them denote histamine and serotonin composition concentrations that showed statistical significance compared to the PBS control. While the results of the preliminary experiment found no statistical significance with the samples. The 4× histamine and serotonin formulation showed promising results and appeared to have the most uniform success across the entire experiment.

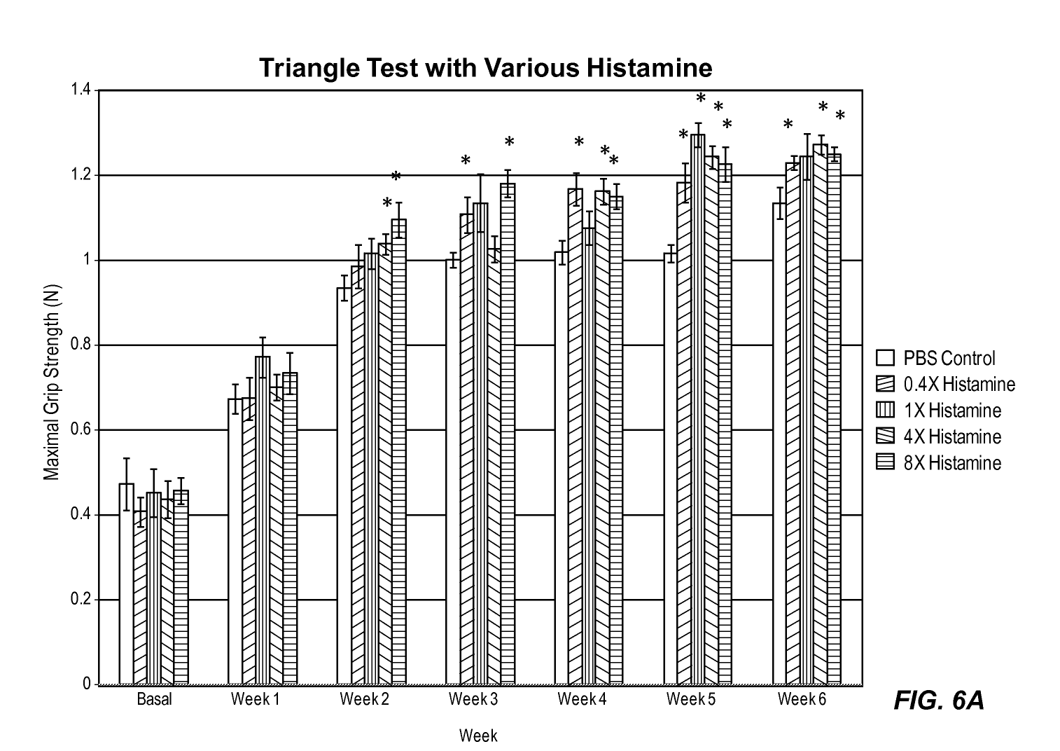

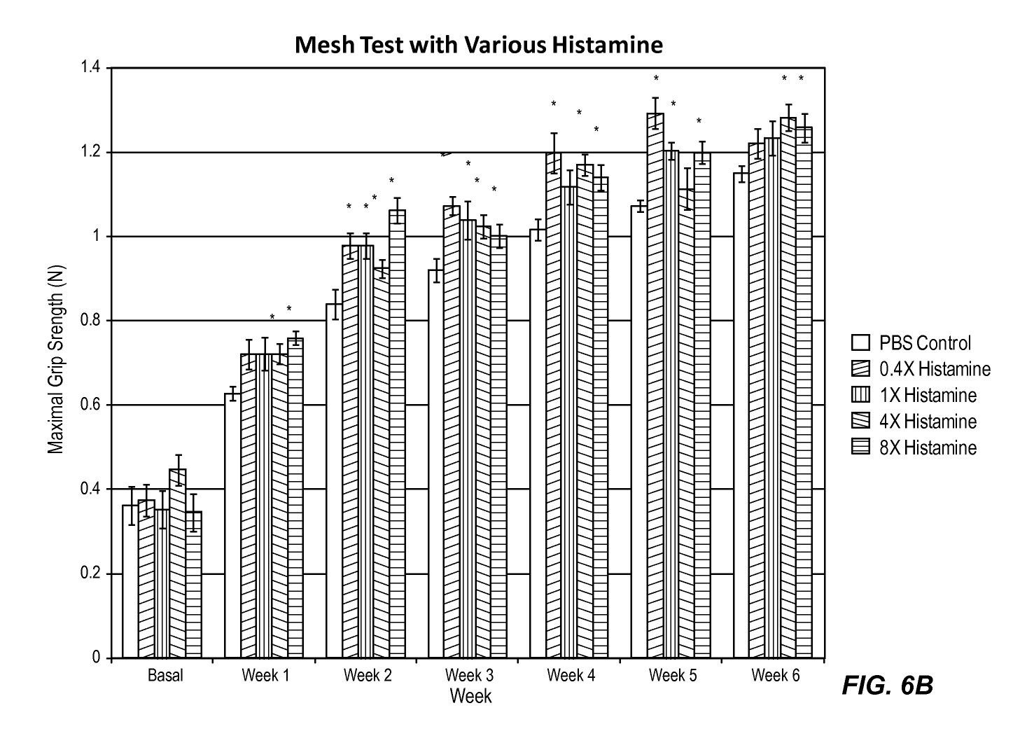

The results of a third experiment testing twice daily administration of different dosages of histamine alone are depicted in FIGS. 6A and 6B. At a 0.1 ml dose the 0.4× histamine formulation contains 8.8 mcg histamine per dosage; the 1× histamine formulation contains 22 mcg histamine per dosage; the 4× histamine contains 88 mcg per dosage and the 8× histamine contains 176 mcg per dosage. The bars noted with an * indicate statistical significantly improved results over the control group which received PBS indicating that administration of histamine alone improves performance in both the Triangle and Mesh tests.

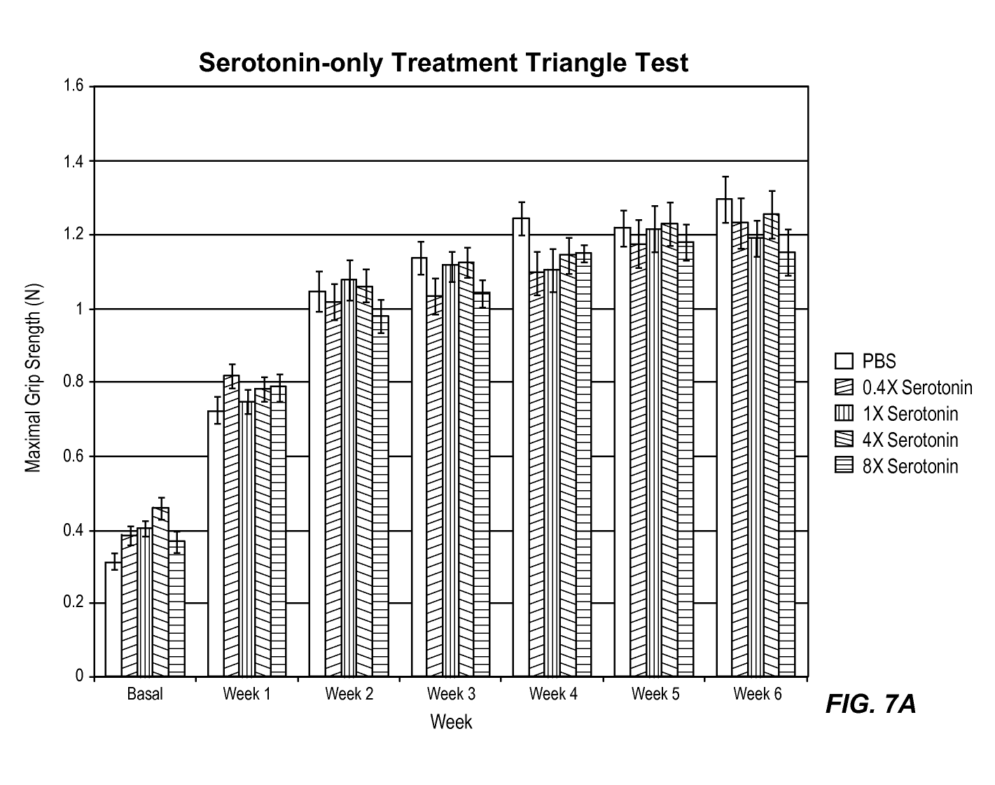

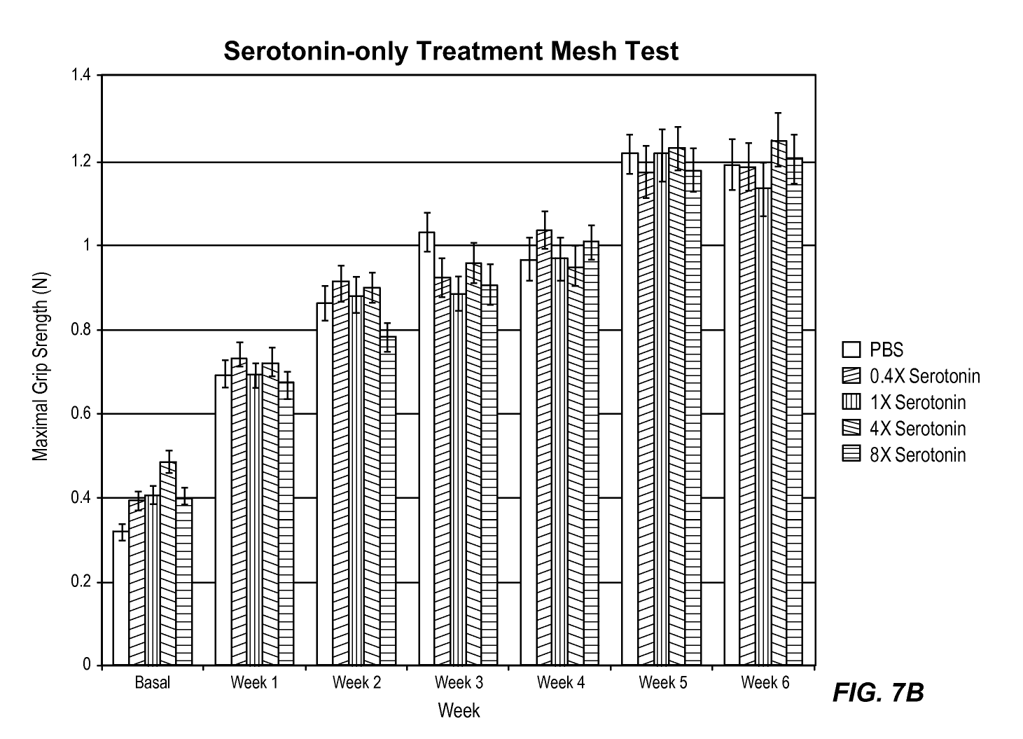

The results of a fourth experiment testing twice daily administration of different dosages of serotonin along are depicted in FIGS. 7A and 7B. At a 0.1 ml dose the 0.4× serotonin formulation contains 16 mcg serotonin per dosage; the 1× serotonin formulation contains 40 mcg serotonin per dosage; the 4× serotonin formulation contains 160 mcg of serotonin per dosage and the 8× serotonin composition contains 320 mcg of serotonin per dosage. The results indicate no statistically significant differences in the grip strength tests between the serotonin test compositions and the PBS controls.

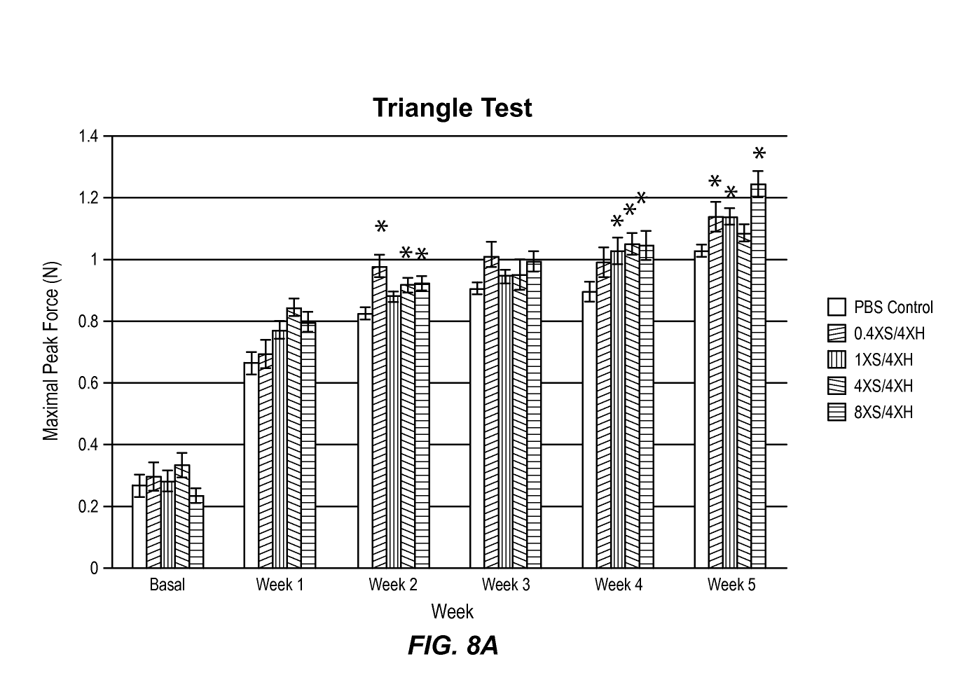

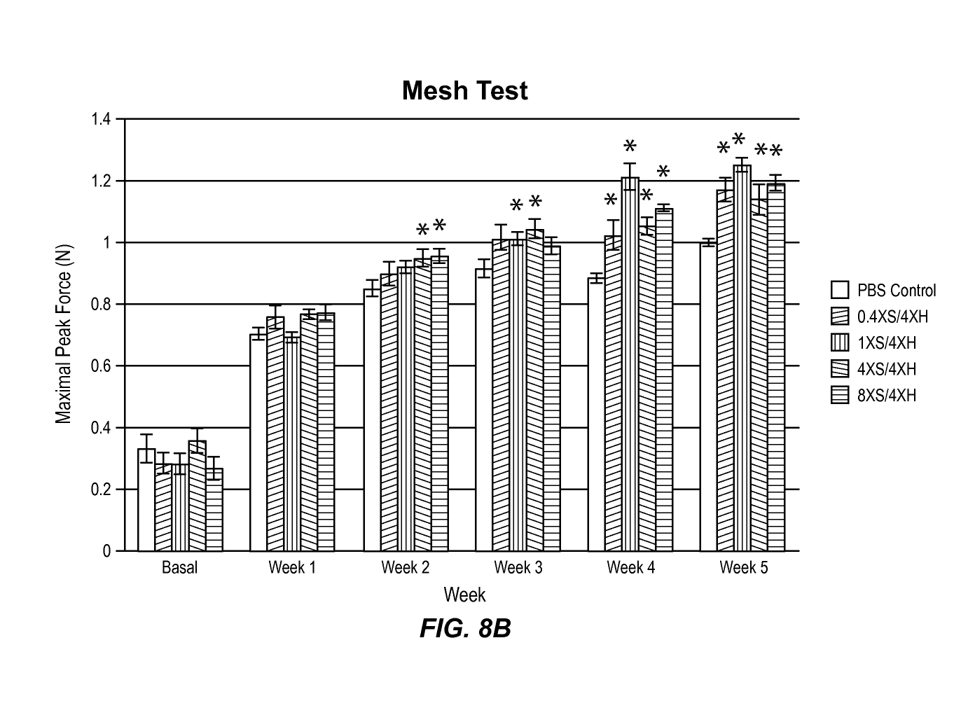

The results of a fifth experiment testing twice daily administration of various test compositions in which the dosage of serotonin was varied are depicted in FIGS. 8A and 8B. At a 0.1 ml dose the 0.4× S/4×H formulation contains 16 mcg serotonin and 88 mcg histamine per dosage; the 1×S/4×H formulation contains 16 mcg serotonin and 88 mcg histamine per dosage; the 0.4×S/4×H formulation contains 40 mcg serotonin and 88 mcg histamine per dosage; the 4×S/4×H formulation contains 160 mcg serotonin and 88 mcg histamine per dosage; and the 8×S/4×H formulation contains 320 mcg serotonin and 88 mcg histamine per dosage. The bars noted with an * indicate statistical significantly improved results over the control group which received PBS indicating that administration of the serotonin and histamine formulations improve performance in both the Triangle and Mesh tests.

### Example 4

According to this example, a subject suffering from Myotonic Dystrophy Type 2 (Proximal Myotonic Myopathy) was treated with the histamine and serotonin compositions of the invention.

Specifically, a 57 year old male diagnosed five years previously with Myotonic Dystrophy Type 2 was seen by a doctor with complaints of weakness in Right and Left legs causing him to have difficulty standing up from sitting position. A genetic test confirmed the diagnosis for Myotonic Dystrophy Type 2 which also affected the subject's sibling.

After baseline testing to assess muscle strength the subject was treated by sublingual administration four times daily of one drop of a solution comprising 4.8×10⁻³ mg histamine and 0.08 milligram serotonin. A baseline testing was done to assess muscle strength.

Assessments on a scale of 1 to 10 were as follows with 10 being the worst, and 0, indicating no symptoms present.

| | After One month | After Three Months |
|---|---|---|
| (1) walking up and down | 4/5 | 3 |
| stairs from first to second | | |
| floor 3 times. | | |
| Lower muscles not | | |
| compromised but subject | | |
| experienced shortness of | | |
| breath | | |
| (2) Toe rises 50 times | 6/7 | 3 |
| (3) Grab ankle and kick | 5/6 | 2 |
| out 10 times | | |
| (4) Grab knee and kick out | 1 | 0 |
| 10 times | | |
| (5) Standing up from | 8/9 | 6/7 |
| sitting in chair 10 times | | |
| (6) Getting up from the | 8 | 4 |
| floor to stand 10 times | | |
| (7) Subject's self | 6 | 3 |
| assessment: “Where are | | |
| you in feeling your muscle | | |
| strength is today” | | |

The overall changes were showing 1-3 points lower (being improved) on each prior test scores. The subject stated that after three months of treatment he felt more energy, and noticed the things that were most difficult for him to do in muscle strength were easier now. The ability to rise from his chair without needing to push off each time, and to get up from the floor to standing without holding on to furniture each time, made a big difference in the subject's feeling of “well being” to see such improvement. These improvements were also reported by the subject's wife and the subject was able to do extensive cross country travel after two months of treatment.

### Example 5

According to this example, a 63 year old female subject suffering from the manifesting carrier state of Duchenne's Muscular Dystrophy was treated by sublingual administration four times daily of one drop of a solution comprising 4.8×10⁻³ mg histamine and 0.08 milligram serotonin for a period of six weeks. The subject reported improved energy and stamina as well as of increased right lower extremity muscle strength as verified by her physical therapist.

### Example 6

According to this example, a five year old boy suffering from Duchenne's Muscular Dystrophy was treated by sublingual administration four times daily of one drop of a solution comprising 4.8×10⁻³ mg histamine and 0.08 milligram serotonin. The subject is reported to be able to jump and lift both feet off the ground which he had not been able to do previously. In addition, he could stand on one leg for 10-12 seconds which he had previously been unable to do.

Numerous modifications and variations in the practice of the invention are expected to occur to those skilled in the art upon consideration of the preferred embodiments. The only limitations which should be placed upon the scope of the invention are those which appear in the appended claims.

What is claimed is:

1. A method for treating muscle weakness resulting from a disease state or injury in a subject suffering therefrom comprising the step of: administering a composition comprising histamine an amount effective to alleviate muscle weakness in the subject suffering therefrom.

2. The method of claim 1, wherein said composition comprises from about 4×10⁻¹ to about 4×10⁻⁵ mg of histamine.

3. The method of claim 1, wherein said composition comprises from about 4×10⁻² to about 4×10⁻⁴ mg of histamine.

4. The method of claim 1, wherein said composition comprises about 1×10⁻³ mg of histamine.

5. The method of claim 1, wherein the histamine used is in a soluble salt.

6. The method of claim 1, wherein said composition further comprises from about 0.2 mg to about 2×10⁻⁶ mg of serotonin (5-hydroxytryptamine).

7. The method of claim 1, wherein said composition further comprises 2×10⁻² mg to about 2×10⁻⁴ mg of serotonin.

8. The method of claim 1, wherein said composition further comprises about 2×10⁻³ mg of serotonin.

9. The method of claim 1, wherein said composition is administered to a patient in a single dose of about 0.05 cc in a pharmaceutically acceptable carrier.

10. The method of claim 9, wherein multiple daily doses of said composition are administered to the patient.

11. The method of claim 9, wherein the composition is administered to a patient sublingually.

12. The method of claim 1, wherein said composition comprises 4×10⁻² to about 4×10⁻⁴ mg of histamine and from about 2×10⁻² mg to about 2×10⁻⁴ mg of serotonin.

13. The method of claim 1, wherein said composition comprises about 0.08 mg of histamine and about 4×10⁻³ of serotonin.

14. The method of claim 1, wherein the disease state or injury is selected from the group consisting of muscular dystrophy, post-polio syndrome, multiple organ dysfunction syndrome, myasthenia gravis, chronic fatigue syndrome, crush injury and inflammatory myopathy.

15. The method of claim 1 wherein the disease state is muscular dystrophy.
